(19) United States
(12) Patent Application Publication
(10) Pub. No.: US 2011/0192421 A1
(43) Pub. Date: Aug. 11, 2011

(54) COMPOSITIONS FOR DEGREASING HARD SURFACES

(75) Inventors: Joaquin Bigorra Llosas, Sabadell (ES); Javier Raya, Sant Vicenç dels Horts (ES); Ramon Valls, Barcelona (ES); Carles Estevez, Sitges (ES); Lidia Galiá, Girona (ES); Josep Castells, Montmeló (ES)

(73) Assignees: COGNIS IP MANAGEMENT GMBH, Düsseldorf (DE); INSTITUT UNIV. DE CIÉNCIA I TECNOLOGIA, S.A., Mollet del Vallés (ES)

(21) Appl. No.: 12/988,414

(22) PCT Filed: Apr. 9, 2009
(86) PCT No.: PCT/EP2009/002667

§ 371 Date: Oct. 18, 2010

(30) Foreign Application Priority Data
Apr. 19, 2008 (EP) EP08007673

(51) Int. Cl. B08B 3/00; C07C 233/02; C11D 3/32

(52) U.S. Cl. 134/10; 564/215; 510/245

(57) ABSTRACT

The invention relates to degreasing compositions comprising at least one dialkyl amide of formula (I), R¹CO—NR²R³, in which R¹CO represents a linear or branched, saturated or unsaturated, aliphatic or aromatic, optionally hydroxysubstituted acyl group having 2 to 56 carbon atoms, and R² and R³ independently represent alkyl groups having 1 to 6 carbon atoms. The compositions are useful for degreasing hard surfaces, particularly metal surfaces.

### CROSS-REFERENCE TO RELATED APPLICATIONS

This application is the U.S. National Stage entry under 35 USC §371 of International Application no. PCT/EP2009/002667, filed Apr. 9, 2009, which claims priority from European Patent Application no. EP08007673, filed Apr. 19, 2008, both of which are incorporated herein in their entireties.

### FIELD OF THE INVENTION

The present invention is related to the area of metal treatment and refers to new degreasing compositions, a process for degreasing hard surfaces and the use of so-called “green” solvents for degreasing operations.

### BACKGROUND OF THE INVENTION

In ordinary metal processing metal parts are greased to avoid the corrosion process during their manufacture, storage and transport. Since such grease is incompatible with subsequent metal processing stages, a cleaning step to remove the metal protector is inevitable. Over the past few years, one of the major challenges in the area of metal degreasing has been the transition from fully emissive open-top systems based on the use of chlorinated solvents to closed-loop metal-degreasing systems based on low volatile organic chemical (VOC) emission, low toxicity solvents. Alternative chlorinated solvents such as trichloroethanol, chloroform, methyl chloride, CFC-113, HFCs, HCFCs, and other solvents including CO₂ jets, super critical CO₂, semi-aqueous solvents, alkaline cleaning agents, emulsifying detergent-based cleaners, aliphatic hydrocarbon-based solvents, and azeotropic mixtures have been proposed to replace the widely used current industrial standard, namely trichloroethylene. However, none of the proposed alternatives fully satisfies the key industrial needs of the metal finishing sector.

Therefore the object of the present invention has been to develop new compositions which allow metal degreasing operations to be performed in highly variable settings, with metal parts of different size and shape, while simultaneously minimizing diffuse emissions, release of contaminated air during loading and unloading, and solvent release from the cleaned metal parts. The use of these compositions should also avoid the generation of large waste streams, and allow the establishment of an easy and cost effective process for recycling the solvent and rinse water, ultimately delivering metal parts adequately conditioned for immediate use in subsequent steps of the metal finishing process. More particularly, the present invention is directed to replacing known commercial degreasing solvents with new compositions which are more efficient, more safe, and more friendly to the environment.

### DETAILED DESCRIPTION OF PREFERRED EMBODIMENTS

The present invention is directed to new degreasing compositions comprising at least one dialkyl amide according to general formula (I)

R¹CO—NR²R³ (I)

in which R¹CO stands for a linear or branched, saturated or unsaturated, aliphatic or aromatic, optionally hydroxysubstituted acyl group having 2 to 56 carbon atoms, and R² and R³ represent independently from each other alkyl groups having 1 to 6 carbon atoms. Preferred solvents are mixtures of dialkylamides according to general formula (I) in which R¹CO stands for alkyl groups having 6 to 10 carbon atoms and R² and R³ represent methyl groups.

Surprisingly it has been observed that dialkyl amides show a high degreasing efficacy combined with improved eco-toxicological behaviour, regardless of whether the metal surfaces have been protected by solvent-based or cereous metal preservatives containing antioxidants and other additives. In addition, the solvents can be easily rinsed off with water, collected, and then recycled without any additional purification. Replacing well known chlorinated organic degreasing solvents (e.g. trichloroethanol, trichloroethylene, perchloroethylene) by dialkyl amides leads to a more environmentally-friendly process without losing degreasing performance.

### Degreasing Process

Another object of the present invention relates to a method for the degreasing of hard surfaces, characterised in that said surfaces are brought into contact with at least one organic solvent selected from the group consisting of dialkyl amides according to general formula (I),

R¹CO—NR²R³ (I)

in which R¹CO stands for a linear or branched, saturated or unsaturated, aliphatic or aromatic, optionally hydroxysubstituted acyl group having 4 to 56 carbon atoms, and R² and R³ represent independently from each other alkyl groups having 1 to 6 carbon atoms.

### Dialkyl Amides

Dialkyl amides suitable as “green” solvents for conducting the degreasing process can be derived from linear or branched, saturated or unsaturated, aliphatic or aromatic, optionally hydroxysubstituted carboxylic acids having 2 to 56 carbon atoms, as for example caprylic acid, capric acid, capronic (caproic) acid, lauric acid, myristic acid, palmitic acid, palmitoleic acid, stearic acid, isostearic acid, oleic acid, petroselinic acid, linolenic acid, linoleic acid, ricinoleic acid, 12-hydroxystearic acid, arachidonic acid, gadoleic acid, erucic acid, behenic acid and their technical grade mixtures, such as coco fatty acid, tallow fatty acid, and the like. Another group of carboxylic acids useful for the preparation of dialkyl amides is represented by the so-called “dimer acids”, which are obtained by dimerisation or trimerisation of oleic acid. Preferred dialkyl amides are derived from saturated fatty acids having 6 to 10 carbon atoms, or oleic acid. Also suitable are certain hydroxy carboxylic acids, for example, lactic acid, or aromatic carboxylic acids, for example, benzoic acid. The alkyl groups are derived from alcohols having 1 to 6 carbon atoms; therefore, “alkyl” may represent ethyl, propyl, butyl, pentyl or hexyl, but is preferably methyl. Consequently, the preferred dialkyl amides are dimethyl amides of the preferred fatty acids cited above.

As discussed above, typically the hard surfaces are metal surfaces, such as those used in the manufacture of automotive and building components. The dialkyl amides, which are used as so-called “green solvents” serve as degreasing agents in order to remove all greases and stains, and, in particular, the preservatives from the surfaces. This can be done either by dipping the parts into the solvent or, more conveniently, by spraying. Once degreasing has taken place, the dialkyl amides are collected and recycled without purification.

### INDUSTRIAL APPLICATION

As outlined above, dialkyl amides show excellent performance in removing stains, grease and especially preservatives from hard surfaces. Another object of the present invention is therefore directed to the use of dialkyl amides according to general formula (I)

R¹CO—NR²R³ (I)

in which R¹CO stand for a linear or branched, saturated or unsaturated, aliphatic or aromatic, optionally hydroxysubstituted acyl group having 4 to 56 carbon atoms, and R² and R³ represent independently from each other alkyl groups having 1 to 6 carbon atoms, as degreasing agents for hard surfaces.

### EXAMPLES

### Metal Degreasing Procedure

For our evaluation, a comparative method was used, in which removal efficacy (RE) of several alternative solvents is compared with the RE value obtained for the industrial standard degreaser, trichloroethylene.

Removal Efficacy (RE) measures the degree of removal of organic materials (grease and/or solvent) from the surface of metal parts. The removal efficacy screening test involves removing the grease from ten metallic greased pieces by a degreasing process. The standard procedure was performed by bringing the solvent into contact with the metal surface, more particularly by immersion without agitation for 10 minutes in one volume of fresh solvent followed by three consecutive washing cycles by immersion in clean water. The amount of organic material (grease and/or solvent) that was not eliminated by this procedure was determined by direct weight after removal of organic residues from the metallic parts using the standard cleaning procedure with trichloroethylene.

The removal efficacy (RE) for the industry-standard degreasing solvent, CHCl═CCl₂, is between 94-98% depending on the nature of the preservative (Table 1). These RE values were used to compare with the results obtained using solvents of the invention and to determine their effectiveness compared with trichloroethylene.

TABLE 1
Removal Efficacy (%) value for trichloroethylene

| Preservative | A (solvent-based) | B (cereous-based) |
|---|---|---|
| RE (%) | 94.2 | 98.1 |

### Example 1

### Degreasing Studies with Dimethylamide Solvents

The degreasing efficacy of the dimethyl amide (DMA) solvent family was studied. These experiments remove the grease of ten greased pieces according to the procedure described above. This experiment was carried out for two different types of grease, and the results are shown in table 2.

TABLE 2
RE (%) value normalized to trichloroethylene
for dimethyl amide (DMA) solvents

| | Solvent-based | Cereous |
|---|---|---|
| Solvent | preservative | preservative |
| Trichloroethylene | 100.0 | 100.0 |
| Capronic acid dimethylamide | 95.58 | 99.68 |
| Caprylic acid dimethyl amide | 45.71 | 91.87 |
| Caprinic acid dimethyl amide | 23.03 | 86.66 |
| Benzoic acid dimethyl amide | 40.34 | 66.27 |
| Lactic acid dimethyl amide | — | 56.68 |
| Oleic acid dimethylamide | — | 65.23 |

### Example 2

### Recovery and Regeneration of Solvent

In order to have an economically viable process, the degreasing solvent must be able to be used several times without any prior purification. For this reason reusability of the solvent capronic acid dimethyl amide (DMA-6) has been studied in both preservatives. The results are outlined in Table 3.

TABLE 3
RE Reusability of DMA-6 (values normalized to trichloroethylene).

| Number of cycles | Solvent based preservative | Cereous preservative |
|---|---|---|
| 1 | 95.02 | 99.68 |
| 2 | 91.94 | 100.47 |
| 5 | 91.94 | 99.94 |
| 9 | — | 99.31 |
| 12 | — | 94.81 |

After 5 cycles with the solvent-based preservative the loss in efficiency was less than 4%. In the case of the cereous preservative, the solvent can be reused 12 times with a loss in efficiency of only 5%.

What is claimed is:

1. A degreasing composition comprising at least one dialkyl amide of formula (I),
R¹CO—NR²R³ (I)
wherein R¹CO represents a linear or branched, saturated or unsaturated, aliphatic or aromatic, optionally hydroxysubstituted acyl group having 2 to 56 carbon atoms, and R² and R³ independently represent alkyl groups having 1 to 6 carbon atoms.

2. The degreasing composition of claim 1, comprising a mixture of dialkyl amides of formula (I) wherein R¹CO represents an acyl group having 6 to 10 carbon atoms, and R² and R³ are methyl.

3. A method for degreasing a hard surface, comprising the step of contacting a hard surface with at least one organic solvent selected from the group consisting of dialkyl amides of formula (I),
R¹CO—NR²R³ (I)
wherein R¹CO represents a linear or branched, saturated or unsaturated, aliphatic or aromatic, optionally hydroxysubstituted acyl group having 2 to 56 carbon atoms, and R² and R³ independently represent alkyl groups having 1 to 6 carbon atoms.

4. The method of claim 3, wherein said dialkyl amides are derived from saturated fatty acids having 6 to 10 carbon atoms.

5. The method of claim 3, wherein said dialkyl amides are derived from oleic acid.

6. The method of claim 3, wherein said dialkyl amides are derived from lactic acid.

7. The method of claim 3, wherein said dialkyl amides are derived from benzoic acid.

8. The method of claim 3, wherein said hard surface comprises a metal surface.

9. The method of claim 3, further comprising the steps of collecting and recyclic said organic solvent without purification, after degreasing has taken place.

10. (canceled)

11. A method of degreasing a hard surface, comprising the step of contacting a hard surface with the degreasing composition of claim 1.

12. The method of claim 11, wherein said hard surface comprises a metal surface.
